(19) United States
(12) Patent Application Publication
(10) Pub. No.: US 2026/0215676 A1
(43) Pub. Date: Jul. 30, 2026

(54) SYSTEMS AND METHODS FOR USE IN INTRAOCULAR LENS METROLOGY

(71) Applicant: Johnson & Johnson Surgical Vision, Inc., Irvine, CA (US)

(72) Inventors: Usiel Malave-Robles, Mayaguez, PR (US); Mihai State, Groningen (NL); John Scott, Inverness (GB); Darren Millington, Coventry (GB)

(21) Appl. No.: 19/039,739

(22) Filed: Jan. 28, 2025

(51) Int. Cl. A61B 3/00; A61F 2/16

(52) U.S. Cl. CPC A61B 3/0016; A61F 2/16

(57) ABSTRACT

A system for use in automated intraocular lens metrology can include a wet cell having a wet cell base including one or more recesses, a wet cell lid, and an insert configured for placement within the wet cell base. The insert can include a first side, and a second side configured to removably engage with the first side. The insert can include one or more first components configured to align with the one or more recesses to inhibit rotation of the insert within the wet cell base, and one or more second components configured to constrain an intraocular lens (IOL) in a horizontal plane. The insert can be configured to hold the IOL between the first and second sides. The system can further include an assembly configured to removably engage with and reduce the conduction of heat through the wet cell.

### FIELD

The present invention generally relates to systems and methods for use in intraocular lens metrology. More specifically, certain embodiments relate to systems and methods for restricting movement of an intraocular lens (IOL) in a wet cell for use in automated intraocular lens metrology.

### BACKGROUND

The ISO Standard 11979-2 requires an IOL be tested according to metrics under the Modulation Transfer Function (MTF) to ensure the IOL satisfies certain optical parameters. The MTF measurements are typically taken using a wet cell with specific dimensions along the optical plane (z-axis). The position of the IOL in the wet cell can impact the MTF results. Traditional wet cells used for taking MTF measurements, such as wet cell 101 shown in FIG. 1, are not configured in such a way as to provide stable lens management within an automated manipulation system.

Accordingly, alternative systems and methods for use in IOL metrology would be useful.

### SUMMARY

The present invention is directed to IOL metrology. In an exemplary application, the systems and methods herein may be configured for conducting automated IOL metrology.

An example system is provided for use in automated intraocular lens metrology. The system can include a wet cell having a wet cell base including one or more recesses, a wet cell lid, and an insert configured for placement within the wet cell base. The insert can include a first side, and a second side configured to removably engage with the first side. The insert can include one or more first components configured to align with the one or more recesses to inhibit rotation of the insert within the wet cell base, and one or more second components configured to constrain an intraocular lens (IOL) in a horizontal plane. The insert can be configured to hold the IOL between the first and second sides. The system can further include an assembly configured to removably engage with and reduce the conduction of heat through the wet cell. The assembly can include an interface and an insulating layer.

Another example system is provided for use in automated intraocular lens metrology. The system can include a wet cell configured to hold a liquid and having a wet cell base including one or more recesses, a wet cell lid, and an insert configured for placement within the wet cell base. The insert can include a first side, and a second side configured to removably engage with the first side. The insert can further include one or more first components configured to align with the one or more recesses to inhibit rotation of the insert within the wet cell base, and one or more second components configured to constrain an intraocular lens (IOL) in a horizontal plane. The insert can be configured to hold the IOL between the first and second sides when the first and second sides are engaged. The weight of the first side can be greater than the weight of the second side to prevent floating of the insert inside the wet cell when the wet cell holds the liquid.

An example method is provided for conducting automated intraocular lens metrology. The method can include engaging an intraocular lens (IOL) with a first side of an insert, and engaging a second side of the insert with the first side such that the IOL is held between the first and second sides. The insert can include one or more first component configured to constrain the IOL in a horizontal plane. The method can further include placing the insert into a wet cell base of a wet cell such that the second side of the insert faces a first direction within the wet cell base and the first side of the insert faces a second direction within the wet cell base, restricting the movement of the IOL within the wet cell by the configuration of the insert. The method can further include engaging a wet cell lid with the wet cell base, and automatically measuring one or more features of the IOL.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of a traditional wet cell found in the prior art.

FIG. 2 is an illustration of an example system for use in automated intraocular lens metrology according to aspects of the present invention.

FIGS. 3A-3C are illustrations of top (A), perspective (B), and bottom (C) views of an example component of the example system of FIG. 1 according to aspects of the present invention.

FIGS. 3D-3E are illustrations of side (D) and front (E) cross-sectional views of the example component of FIGS. 3A-3C, according to aspects of the present invention.

FIG. 4 is an illustration of an example component of the example system of FIG. 1 according to aspects of the present invention.

FIG. 5 is an illustration of an example component of the example system of FIG. 1 according to aspects of the present invention.

FIG. 6 is an illustration of an example component of the example system of FIG. 1 according to aspects of the present invention.

FIG. 7 is an illustration of example components of the example system of FIG. 1 according to aspects of the present invention.

FIG. 8 is an illustration of example components of the example system of FIG. 1 according to aspects of the present invention.

FIG. 9 is an illustration of example components of the example system of FIG. 1 according to aspects of the present invention.

FIG. 10 is an illustration of an example component of the example system of FIG. 1 according to aspects of the present invention.

FIG. 11 is a flowchart of an exemplary method for conducting automated intraocular lens metrology according to aspects of the present invention.

### DETAILED DESCRIPTION

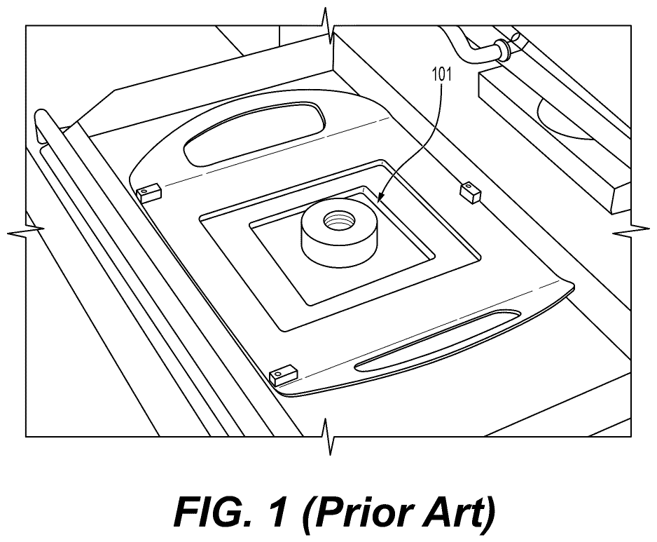

Traditional wet cell designs, such as the wet cell 101 shown in FIG. 1, can present challenges in providing stable lens management within an automated manipulation system. For example, traditional wet cell designs can include components having respective shapes, weights, and/or engagement mechanisms that can lead to the IOL shifting or moving during automated manipulation. Typically, these traditional wet cells require placing the IOL in a holder to take the MTF measurements. During automated water cell manipulation, the holder can start to float at different elevations within the wet cell resulting in inaccurate results and/or measurement variability. Further, when transporting the well cell components, traditional material handling systems can themselves transfer heat to the wet cell, which can change the optical properties of the IOL and of the water required for proper inspection. Accordingly, the example systems and methods described herein overcome these and other challenges by providing a wet cell design configured to restrict movement of the IOL within the wet cell during the automated manipulation to retain measurement accuracy and reduce any potential variability while still complying with the ISO 11979-2 Standard. Additionally, the example systems and methods described herein are configured for conducting fully automated handling, measurement, and drying of the IOL through the use of one or more wet cell components and/or features (e.g., size, shape, etc.) that allow the wet cell to be connected to, manipulated by, and/or transported by automated devices and processes. Finally, the example systems and methods described herein are configured to reduce heat conduction through the wet cell caused by an external material handling system.

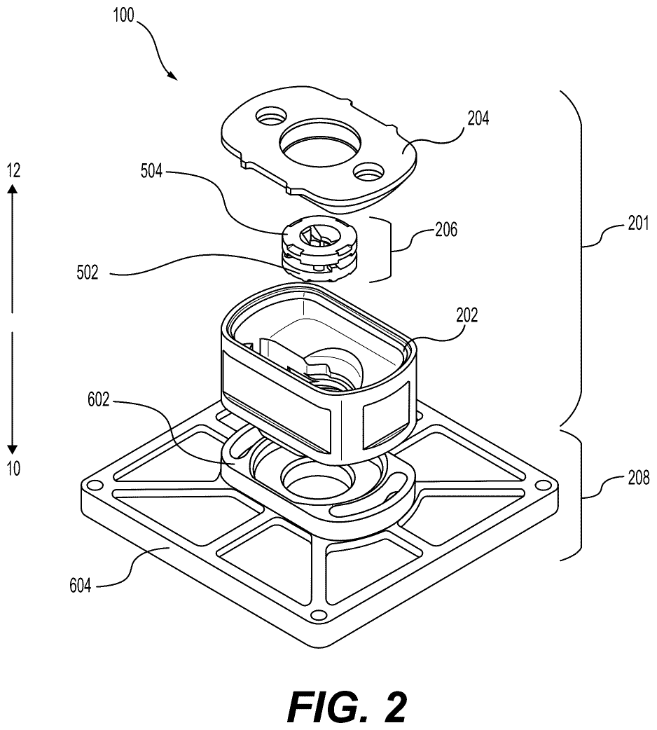

Various example systems and methods are presented herein. Features from each example are combinable with other examples as understood by persons skilled in the pertinent art. FIGS. 2, 3A-3E, and 4-9 provide example components of a system for conducting automated IOL metrology and are discussed simultaneously herein.

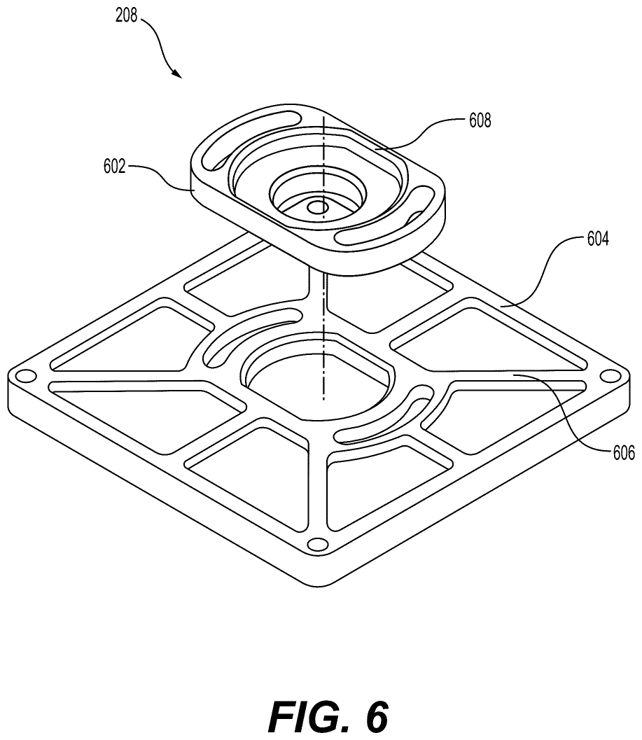

FIG. 2 is an example system 100 used for conducting automated IOL metrology. The system 100 can include a wet cell 201, and an assembly 208. The wet cell 201 can include a wet cell base 202, a wet cell lid 204, and an insert 206. As particularly shown in FIGS. 7-9 and discussed further below, the insert 206 can be configured to hold an IOL 205 between a first side 502 and a second side 504 for conducting automated IOL metrology.

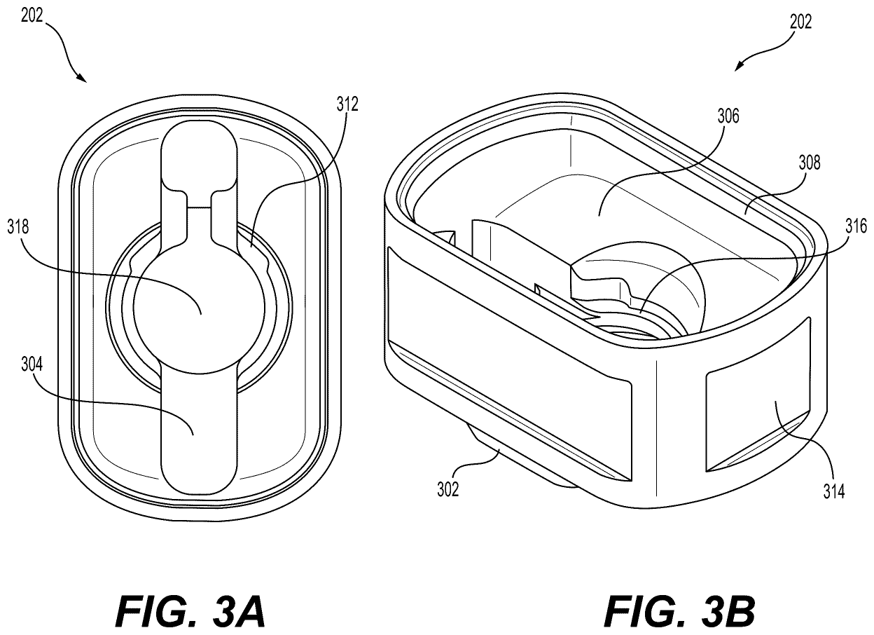

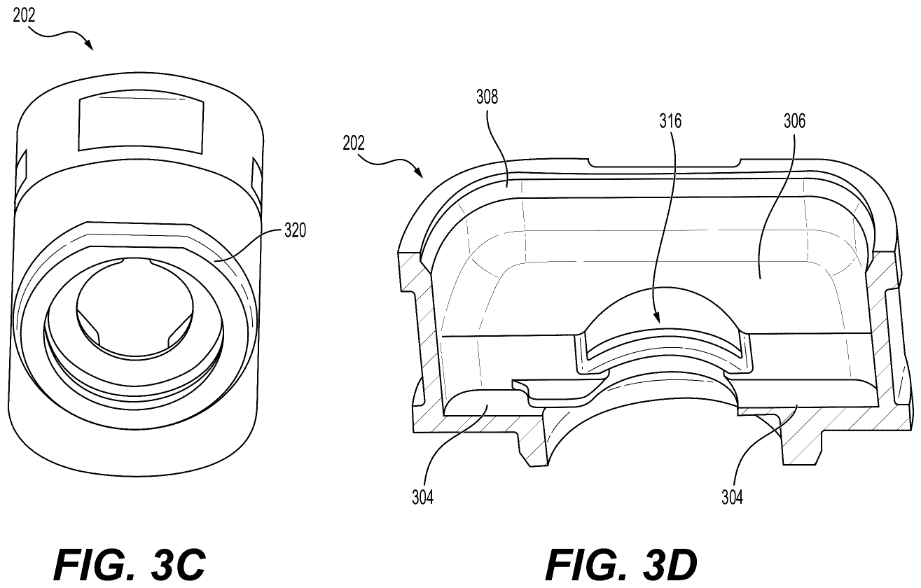

FIGS. 3A, 3B, and 3C respectively show top, perspective, and bottom views of an example wet cell base 202 of the system 100. FIGS. 3D and 3E respectively show side and front cross-sectional views of the example wet well base 202 of the system 100. As particularly shown in FIG. 3B, the wet cell base 202 can include one or more locating features 302 configured to aid in determining the orientation of the wet cell 201 and/or to engaging with one or more mating features of an assembly 208, as further discussed below. As particularly shown in FIGS. 3A and 3D, the wet cell base 202 can also include one or more channels 304 that aid in dosing water (e.g., deionized water (“DI water”)) into the wet cell 201. For example, the channel(s) 304 provide a position to dose and/or purge the water into and/or out of the wet cell 201. The channel(s) 304 can also provide clearance for a tool, such as a gripper, to assemble and/or disassemble the insert 206 within the wet cell 201, as further discussed below. As particularly shown in FIGS. 3D and 3E, the wet cell base 202 can also include one or more surfaces 306 that can be sloped to help prevent water from becoming trapped in the wet cell base 202 during purging of the wet cell 201.

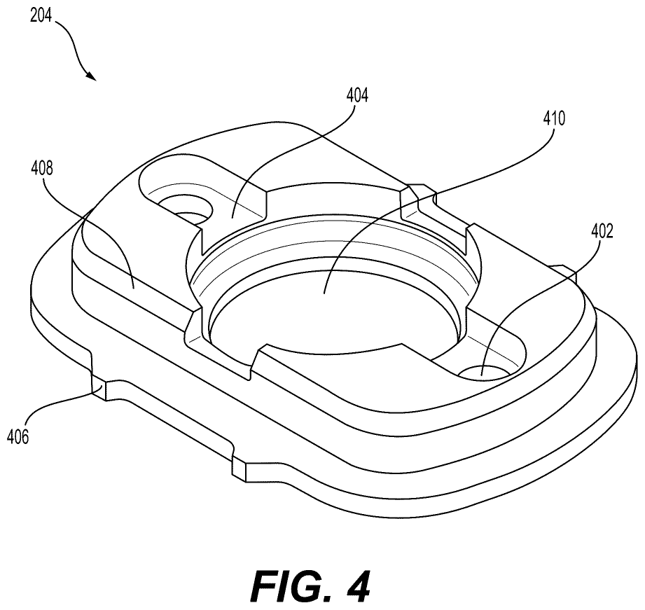

The wet cell base 202 can also include one or more base chamfers 308 that help engage or align the wet cell base 202 with the wet cell lid 204, as further discussed below. Any slight misalignment of the wet cell base 202 with the external material handling system (e.g., on the order of about 20 microns) can impact the operation of the system 100. As such, the wet cell base 202 can also include a lower base chamfer 320 (FIG. 3C) that can also help to engage or align the wet cell base 202 with an external material handling system (e.g., during a robotic placement operation), as further discussed below. The wet cell base 202 can also include one or more boss features (e.g., location feature(s) 302) to help engage or align the wet cell base 202 with an external material handling system, as further discussed below. The wet cell base 202 can also include one or more recesses 312 configured to engage with one or more lobes 514 of the insert 206 (FIG. 5) to reduce rotation of the insert 206 when the insert 206 is placed in the wet cell base 202, as further discussed below.

The wet cell base 202 can also include one or more wall recesses 314 to provide gripping locations for manipulating and/or transporting the wet cell 201. The wet cell base 202 can also include an opening 316 configured to engage with the insert 206 when the insert 206 is placed in the wet cell base 202, as further discussed below. The wet cell base 202 can include a window hole 318 for holding a transparent material, such as glass, for viewing into the wet cell 201 (e.g., for conducting cosmetic inspection) and/or for performing MTF. The glass can have a specific refraction index for taking the MTF measurements, and can have a diameter of between approximately 22 to 30 (e.g., 26) millimeters (mm), and a thickness of between approximately 3 to 9 (e.g., 6) mm. The refraction index, thickness, and/or parallelism of the glass can be adjusted to satisfy applicable ISO standard(s).

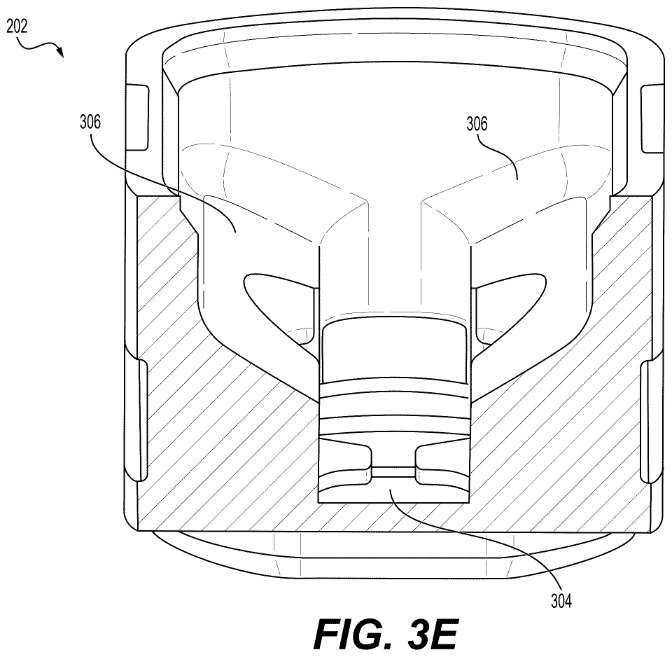

FIG. 4 provides an example wet cell lid 204 of the system 100. The wet cell lid 204 can include one or more ports 402 configured to aid in dosing and/or purging of water into the wet cell 201 while the wet cell lid 204 is engaged with the wet cell base 202. The port(s) 402 can be counterbored providing a bund to reduce the chance of spillage in the event of over-dosing. The port(s) 402 can also be configured of a size to accommodate a temperature sensor (e.g., a PT100 sensor) to allow for temperature monitoring of the wet cell 201 while the wet cell base 202 and wet cell lid 204 are engaged. The wet cell lid 204 can also include a window hole 410 for holding a transparent material, such as glass, for viewing into the wet cell 201 and/or for performing MTF. The glass can have a specific refraction index for taking the MTF measurements, and can have a diameter of between approximately 22 to 30 (e.g., 26) millimeters (mm), and a thickness of between approximately 3 to 9 (e.g., 6) mm. The refraction index, thickness, and/or parallelism of the glass can be adjusted to satisfy applicable ISO standard(s). The wet cell lid 204 can also include one or more slots 404 to allow air to escape the surface of the glass during dosing of the wet cell 201. The slot(s) 404 can also help protect the glass from any potential damage when the wet cell 201 or wet cell lid 204 are placed on a work surface. The wet cell lid 204 can also include one or more protruding edges 406 (e.g., proud edges) configured to aid in manipulating the wet cell lid 204 independent of the wet cell 201. The wet cell lid 204 can also include one or more lid chamfers 408 configured to aid in engaging the wet cell lid 204 with the wet cell base 202 such that the two components of the wet cell 201 align and fit together appropriately.

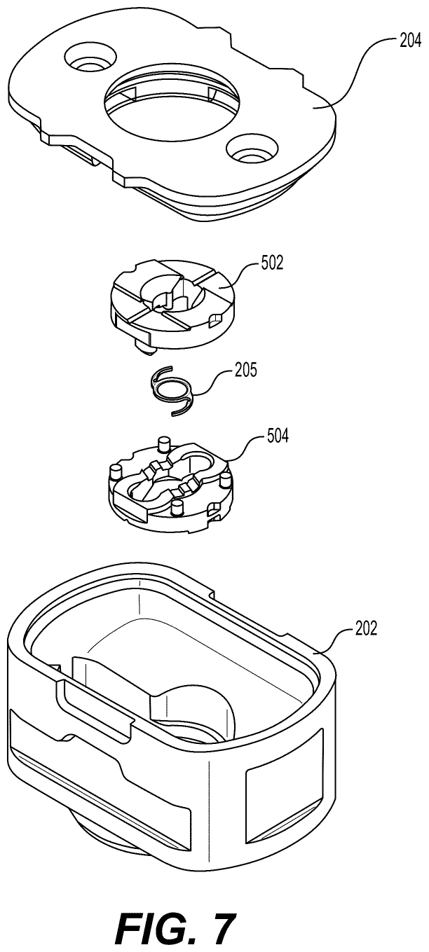

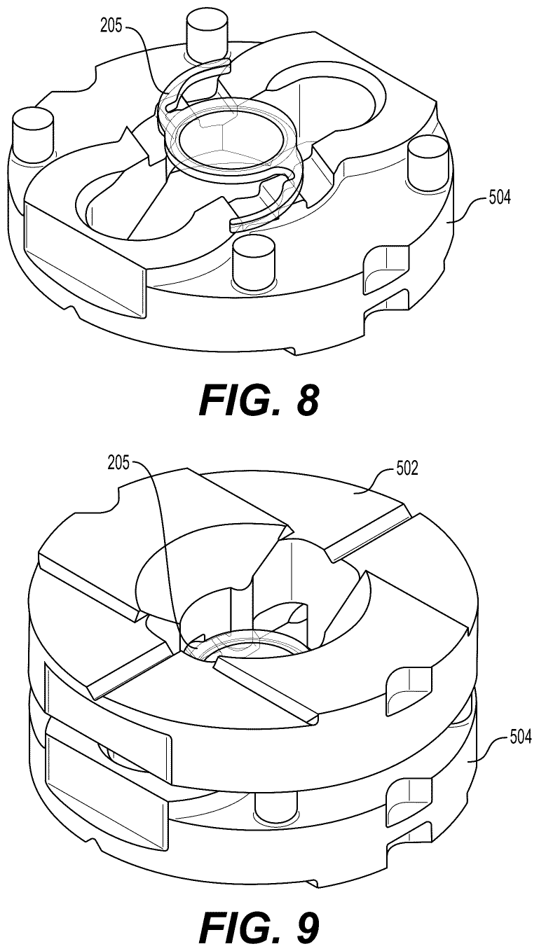

FIG. 5 provides an example insert 206 of the system 100. The insert 206 can be configured to restrict movement of the IOL within the wet cell 201 to reduce variability and inaccuracies in MTF measurements. As shown, the insert 206 can include a first side 502 and a second side 504. The first and second sides 502, 504 are configured to removably engage with each other such that an IOL can be placed between the first and second sides 502, 504 and be held in place in such position when the insert 206 is placed in the wet cell 201. For example, as particularly shown in FIGS. 8 and 9, the IOL 205 can first be placed on top of the second side 504 (FIG. 8), and then the first side 502 placed on top of the IOL 205 and the second side 504 (FIG. 9). The first and second sides 502, 504 are configured to engage with one another via one or more of their respective components described herein to restrict movement of the IOL 205. The insert is configured such that the IOL can be placed between the first and second sides 502, 504 via an automated process (e.g., via a pick-and-place machine). The first and second sides 502, 504 can be made of respective materials such that the weight of the first side 502 is greater than the weight of the second side 504. For example, the first side 502 can be made of stainless steel, while the second side 504 can be made of polyetheretherketone (PEEK). In some embodiments, both the first and second sides 502, 504 can be made of a metal. A benefit of such material composition of the first and second sides 502, 504 is that the insert 206 can be placed in the wet cell 201 with the first (heavier) side 502 facing downward to inhibit the insert 206 from floating within the wet cell 201 when the wet cell 201 is dosed with water, as further discussed below.

The insert 206 can include one or more first insert chamfers 506, such as on the second side 504, to assist in directing the flow of water into the insert 206 to surround the IOL therein. The first insert chamfer(s) 506 also minimize the presence of air bubbles around the IOL and hence enable a stable placement with increased consistency of the MTF measurements. The insert 206 can also include one or more second insert chamfers 508 around the perimeter of the insert 206 (e.g., around the first and/or second sides 502, 504) to provide locations for gripping and/or manipulating the insert 206. The insert 206 can include one or more posts 510 to align first and second sides 502, 504 such that they can engage with one another, as discussed herein.

The insert 206 can include one or more standoffs 512 configured to create a gap G between the first and second sides 502, 504 when the first and second sides 502, 504 are engaged. The gap G can be configured at any fixed distance to seat the first and second sides 502, 504 together without crushing or damaging the IOL. The gap G also allows for maximum airflow between the first and second sides 502, 504 for drying, while still supporting the IOL.

The insert 206 can also include one or more lobes 514 around the perimeter of the insert 206 (e.g., around the second side 504) to align the insert 206 with the recesses 312 of the wet cell base 202 when the insert 206 is placed in the wet cell base 202 (e.g., via the opening 316), as discussed above. The insert can include one or more components 516, such as haptic pegs, configured to constrain the IOL in the horizontal place between the first and second sides 502, 504 and within the wet cell 201.

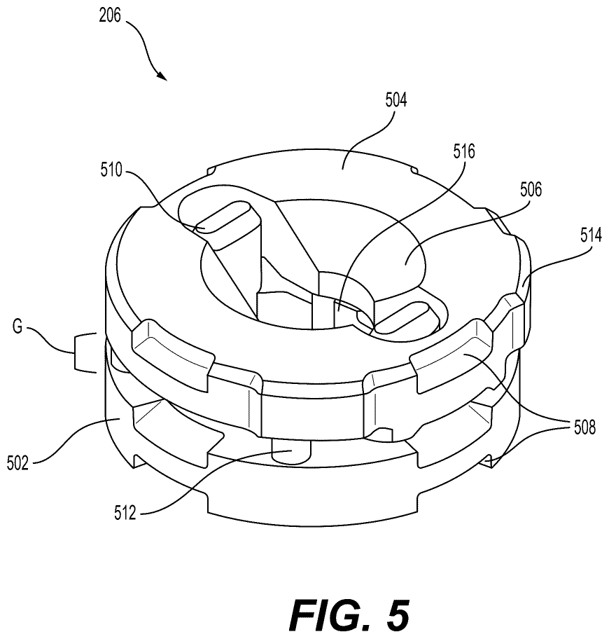

FIG. 6 provides an example assembly 208 of the system 100. A purpose of the assembly 208 is to reduce heat conduction through the wet cell 201 caused by an external material handling system, such as a commercial planar motor conveyor marketed under the “XPlanar” brand by the Italian company BeckhoffAutomation S.r.l., with registered office in Limbiate (“XPlanar system”). Such external handling system can be required to transport the wet cell; however, can cause the transfer of heat to the wet cell which can change the optical properties of the IOL and the water required for inspection within the wet cell. Any heat generated via the external handling system can also increase when the speed of the external system increases, thus insulation can be required when running the external handling system at desired speeds.

Given these challenges, the assembly 208 can include an interface 602 configured to removably engage with the wet cell 201 and an insulating layer 604. The interface 602 can be made of one or more materials, such as stainless steel, to provide durability and robust mating of the interface 602 with the wet cell 201. The interface 602 can include one or more components, such as interface chamfer(s) 608, to allow the interface 602 to properly mate with one or more complementary features of the wet cell base 202 (e.g., locating feature(s) 302) and/or the insulating layer 604. In some embodiments where the assembly 208 does not include a separate interface 602, the insulating layer 604 may include one or more complementary features such that the wet cell base 202 properly mates directly to the interface 602. The insulating layer 604 can be made of one or more materials, such as PEEK, to provide sufficient insulation from the heat conducted via the external handling system. The insulating layer 604 can also include one or more features, such as offset ribs 604, to increase or enhance the heat insulation through the insulating layer 604.

In some examples, the wet cell 201 and the interface 602 make up between approximately 45 to 65 weight percent (e.g., approximately 55 weight percent) of the maximum loading of the external material handling system. In some examples, the wet cell 201, the assembly 208, and a dosage of water used in the wet cell 201 together make up between approximately 73 to 93 weight percent (e.g., approximately 83 weight percent) of the maximum loading of the external material handling system. Given these respective weights of the various components of the system 100, the external material handling system is able to transport the wet cell 201 and/or the system 100 at any desired and/or maximum speed without the wet cell exceeding any lifting capacity of the external material handling system.

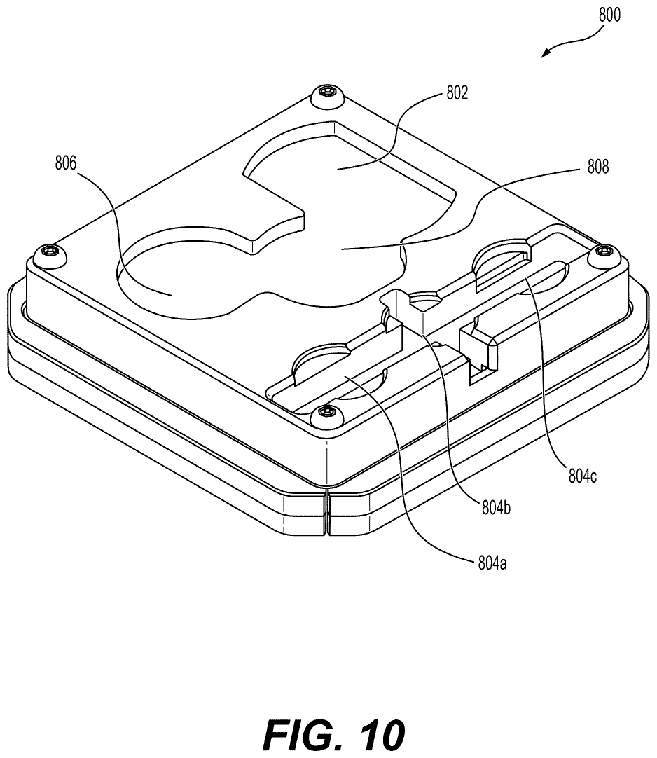

FIG. 10 provides another example assembly 800 of the system 100. In some embodiments, assembly 800 can be used as an alternative to assembly 208 (FIG. 6). Assembly 800 may provide one or more of the same or similar functions as assembly 208, as discussed above. In some embodiments, assembly 800 may be configured such that it can be transported or conveyed along a plurality of locations or stations along an external material handling system as part of a predefined sequence of movements and/or actions.

In some embodiments, the external material handling system includes a plurality of tiles (e.g., 23 tiles) and movers (e.g., 18 movers). On each mover is a set of tooling designed to hold the components of the wet cell 201, including the IOL 205, in different positions, orientations, and states of assembly. On the underside of the tiles are cooling blocks designed to dissipate and remove heat produced by the tiles through conductive and water cooling. The blocks are used to locate and fix the tiles to the cell frame top plates. Around the perimeter of the external material handling system is a series of guarding designed to contain movers within the designated material handling system area should a fault condition arise.

In some embodiments, the external material handling system provides consistent output from interacting modules, and is designed with one primary steady state indexing sequence. However, should there be an interruption from other depended modules, for example a delay in output or a measured parameter that is out of range and requires re-test, the mover routes can adapt. This results in the material handling system, at any given time, seeking the most efficient route to resolve a disruption and recover back to a specific operating condition, reducing cycle time and operator intervention.

In some embodiments, the material handling system is used for picking and placing the different components of the wet cell 201. For example, a first station along the material handling system may be a hold position where the assembly 800, and any connected components of the wet cell 201, may be held steady. As another example, a second station may be a rotation position where the assembly 800, and any connected components of the wet cell 201, may be rotated. At each station along the material handling system, one or more components of the wet cell 201 may be manipulated in some fashion, such as picked up, placed down, dried, etc.

Hovering time of the material handling system with the tiles is minimised to reduce convective heat transfer between the tile and the mover. Heat build-up in the material handling system could result in transmission to the assembly 800 and thus the utilized deionised water, putting it out of range for IOL testing. The material handling system tooling is designed in such a way as to minimise heat transfer between it and the mover body. It achieves this through minimal area contact and by means of an air gap on the internal face of the tooling. A fully loaded mover containing wet cell components is also well within the designated maximum payload limit of the mover, meaning it works in a comfortable operating window which also aids excessive heat build-up. The system also allows for interaction of modules whilst the movers are levitated, further reducing overall mover contact time.

The tilt function of the material handling system can also be utilized in purging of deionized water. For this operation the mover can tilt at an angle allowing remaining water to run to a sump area of the wet cell body and be removed, allowing for more complete extraction and reduced drying time.

The tooling is also designed so that it contains any residual water that may accumulate on it during constant assembly and disassembly cycles. This stops any water seeping from the mover tooling and migrating onto the tile surfaces.

To conduct the above-described sequences along the material handling system, the assembly 800 may be configured to align or mate with one or more of the individual components of the wet cell 201. For example, as shown in FIG. 10, the assembly 800 may include a first slot 802 configured to hold, e.g., the wet cell lid 204. As shown, the first slot 802 may be configured in a certain shape and/or to have edges that mate or align with complementary features of the wet cell lid 204 such that any rotation or movement of the wet cell lid 204 can be prevented when the wet cell lid 204 is positioned in the first slot 802. The assembly 800 may also include one or more second slots 804 (e.g., 804a, 804b, 804c) configured to hold one or more components of the insert 206 (e.g., the first side 502, second side 504, and/or IOL 205). The second slot(s) 804 may be configured in a certain shape and/or to have edges that mate or align with complementary features of the insert 206 (or components thereof). The assembly 800 may also include a third slot 806 and a fourth slot 808 that each may be configured to hold one or more components of the wet cell 201, such as the wet cell base 202 (with or without the insert 206 placed inside) and/or wet cell lid 204, and may themselves be configured to have a certain shape and/or edges to mate or align with complementary features of the wet cell components.

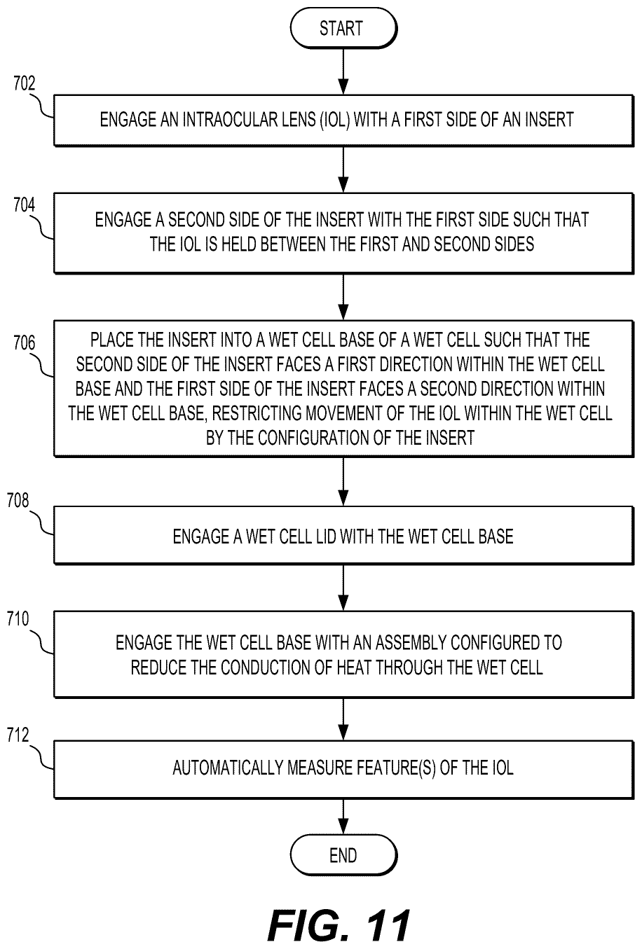

FIG. 11 provides a flowchart of an example method 700 for conducting automated IOL metrology.

In block 702, the method can include engaging an IOL with a first side (e.g., side 504) of an insert (e.g., insert 206), for example, as discussed above with respect to FIG. 8.

In block 704, the method can include engaging a second side (e.g., side 502) with the first side such that the IOL is held between the first and second sides. As discussed herein, the first and second sides can mate with one another via one or more complementary features, such as one or more posts (e.g., 510) that align the first and second sides, and one or more standoffs (e.g., 512) that create a gap between the first and second sides to allow air flow around the IOL without causing damage to the IOL. As discussed herein, the configuration of the first and second sides can also aid in restricting movement of the IOL in the horizontal plane.

In block 706, the method can include placing the insert into a wet cell base (e.g., wet cell base 202) of a wet cell (e.g., wet cell 201) such that the second side (e.g., side 502) of the insert faces a first direction (e.g., downward) within the wet cell base and the first side (e.g., side 504) of the insert faces a second direction (e.g., upward) within the wet cell base. As discussed above, the first and second sides of the insert are respectively made of certain materials such that the second side is heavier than the first side. For example, the first side can be made of PEEK, while the second side made of stainless steel. This difference in weight and positioning of the insert in the wet cell base with the heavier side facing downward, helps to prevent the insert from floating within the wet cell when the wet cell is dosed with water.

In block 708, the method can include engaging a wet cell lid (e.g., wet cell lid 204) with the wet cell base. The wet cell lid can be engaged with the wet cell base via one or more complementary features as discussed herein. For example, the wet cell base can have one or more chamfers (e.g., 308) configured to mate or seat with complementary components on the wet cell lid.

In block 710, the method can include engaging the wet cell base with an assembly (e.g., assembly 208) configured to reduce the conduction of heat through the wet cell. As discussed herein, the assembly can include an insulating layer (e.g., insulating layer 604) and an interface (e.g., interface 602) configured to removably engage with the wet cell and the insulating layer. The insulating layer, for example via one or more offset ribs, can reduce the conduction of heat through the wet cell caused by an external material handling system, such as an XPlanar system.

In block 712, the method can include automatically measuring one or more features of the IOL. That is, once the IOL is positioned between the first and second sides of the insert, the insert placed within the wet cell base, the wet cell lid engaged with the wet cell base, and the wet cell engaged with the assembly, the respective features of each of these components, as discussed herein, restricts the movement of the IOL such that automated IOL metrology can be conducted without the risk of measurement variability and/or inaccuracies.

In some examples, disclosed systems or methods may involve one or more of the following clauses:

Clause 1: A system for use in automated intraocular lens metrology, the system comprising: a wet cell comprising: a wet cell base comprising one or more first recesses; a wet cell lid; and an insert configured for placement within the wet cell base and comprising: a first side; a second side configured to removably engage with the first side; one or more first components configured to align with the one or more first recesses to inhibit rotation of the insert within the wet cell base; and one or more second components configured to constrain an intraocular lens (IOL) in a horizontal plane, wherein the insert is further configured to hold the IOL between the first and second sides; and an assembly configured to removably engage with and reduce the conduction of heat through the wet cell.

Clause 2: The system of clause 1, wherein the first side of the insert comprises stainless steel.

Clause 3: The system of clause 1, wherein the second side of the insert comprises polyetheretherketone (PEEK).

Clause 4: The system of clause 1, wherein the insert comprises one or more standoffs configured to create a gap between the first and second sides when the first and second sides are engaged.

Clause 5: The system of clause 1, wherein the one or more second components comprise one or more haptic pegs.

Clause 6: The system of clause 1, wherein the insert comprises one or more posts configured to align the first and second sides when the first and second sides are engaged.

Clause 7: The system of clause 1, wherein the one or more first components comprise one or more lobes, and wherein the second side of the insert comprises the one or more lobes.

Clause 8: The system of clause 1, wherein the assembly is configured to reduce the conduction of heat through the wet cell from an external material handling system.

Clause 9: The system of clause 1, wherein: the assembly is configured to removably engage with the wet cell base.

Clause 10: The system of clause 1, wherein the assembly comprises one or more offset ribs configured to reduce the conduction of heat through the wet cell.

Clause 11: A system for use in automated intraocular lens metrology, the system comprising: a wet cell configured to hold a liquid and comprising: a wet cell base comprising one or more first recesses; a wet cell lid; and an insert configured for placement within the wet cell base and comprising: a first side; a second side configured to removably engage with the first side; one or more first components configured to align with the one or more first recesses to inhibit rotation of the insert within the wet cell base; and one or more second components configured to constrain an intraocular lens (IOL) in a horizontal plane, wherein the insert is configured to hold the IOL between the first and second sides when the first and second sides are engaged; and wherein the weight of the first side is greater than the weight of the second side to prevent floating of the insert inside the wet cell when the wet cell holds the liquid.

Clause 12: The system of clause 11, wherein the first side of the insert comprises stainless steel.

Clause 13: The system of clause 12, wherein the second side of the insert comprises polyetheretherketone (PEEK).

Clause 14: The system of clause 11, wherein the insert comprises one or more standoffs configured to create a gap between the first and second sides when the first and second sides are engaged.

Clause 15: The system of clause 11, wherein the one or more second components comprise one or more haptic pegs.

Clause 16: The system of clause 11, wherein the insert comprises one or more posts configured to align the first and second sides when the first and second sides are engaged.

Clause 17: The system of clause 11, wherein the one or more first components comprise one or more lobes, and wherein the second side of the insert comprises the one or more lobes.

Clause 18: A method for conducting automated intraocular lens metrology, the method comprising: engaging an intraocular lens (IOL) with a first side of an insert; engaging a second side of the insert with the first side such that the IOL is held between the first and second sides, wherein the insert comprises one or more first components configured to constrain the IOL in a horizontal plane; placing the insert into a wet cell base of a wet cell such that the first side of the insert faces a first direction within the wet cell base and the second side of the insert faces a second direction within the wet cell base, restricting the movement of the IOL within the wet cell by the configuration of the insert; engaging a wet cell lid with the wet cell base; and automatically measuring one or more features of the IOL.

Clause 19: The method of clause 18, wherein: the first direction comprises an upward direction; the second direction comprises a downward direction; and the weight of the second side is greater than the weight of the first side to prevent floating of the insert inside the wet cell when the wet cell holds a liquid.

Clause 20: The method of clause 19, further comprising: engaging the wet cell base with an assembly configured to reduce the conduction of heat through the wet cell from an external material handling system.

Clause 21: The system of clause 1, wherein the wet cell and the interface make up approximately 53 weight percent of a maximum loading of a conveyor system.

Clause 22: The system of clause 1, wherein the wet cell, the assembly, and a dosage of water make up approximately 83 weight percent of a maximum loading of a conveyor system.

Clause 23: A wet cell base of a wet cell, the wet cell base comprising: one or more locating features configured to aid in determining a first orientation of the wet cell; one or more channels configured to aid in dosing water into the wet cell; one or more chamfers configured to aid in engaging the wet cell base with a first component of the wet cell; and one or more first recesses configured to aid in engaging the wet cell base with a second component of the wet cell.

Clause 24: The wet cell base of clause 23, further comprising one or more surfaces configured to prevent the water from becoming trapped in the wet cell.

Clause 25: The wet cell base of clause 24, wherein the one or more surfaces comprise a sloped surface.

Clause 26: The wet cell base of clause 23, wherein the first component comprises a wet cell lid of the wet cell.

Clause 27: The wet cell base of clause 23, wherein the one or more chamfers are further configured to aid in engaging the wet cell base with an assembly, the assembly configured to reduce the conduction of heat through the wet cell from an external material handling system.

Clause 28: The wet cell base of clause 23, wherein the second component comprises an insert of the wet cell, the insert being configured to hold an intraocular lens (IOL).

Clause 29: The wet cell base of clause 28, wherein the one or more first recesses are configured to engage with one or more components of the insert thereby constraining a second orientation of the insert in the wet cell.

Clause 30: The wet cell base of clause 23, wherein the one or more channels are further configured to aid in purging the water out of the wet cell.

Clause 31: The wet cell base of clause 23, wherein the one or more channels are further configured to aid in one or more of assembling and disassembling an insert within the wet cell, the insert being configured to hold an intraocular lens and prevent movement of the IOL within the wet cell.

Clause 32: The wet cell base of clause 23, further comprising: one or more boss features configured to aid in engaging the wet cell base with an assembly configured to reduce the conduction of heat through the wet cell from a material handling system.

Clause 33: The wet cell base of clause 23, further comprising: one or more second recesses configured to aid in one or more of transporting and manipulating the wet cell.

Clause 34: The wet cell base of clause 33, wherein the one or more second recesses are disposed on one or more external surfaces of the wet cell base.

Clause 35: The wet cell base of clause 23, further comprising: an opening 316 configured to engage with an insert of the wet cell, the insert being configured to hold an intraocular lens (IOL).

Clause 36: The wet cell base of clause 35, further comprising a window hole.

Clause 37: The wet cell base of clause 36, wherein the window hole comprises glass.

Clause 38: The wet cell base of clause 37, wherein the glass comprises a diameter of approximately 26 millimeters and a thickness of approximately 6 millimeters.

Clause 39: A wet cell lid of a wet cell, the wet cell lid comprising: one or more ports configured to aid in dosing of water into the wet cell; one or more slots configured to allow air to escape from the wet cell lid; and one or more chamfers configured to aid in engaging the wet cell lid with a wet cell base of the wet cell.

Clause 40: The wet cell lid of clause 39, wherein the one or more ports are further configured to aid in purging the water from the wet cell.

Clause 41: The wet cell lid of clause 39, wherein the one or more ports comprise a counterbored port.

Clause 42: The wet cell lid of clause 39, wherein the one or more ports are further configured to aid in monitoring a temperature of the wet cell.

Clause 43: The wet cell lid of clause 42, wherein the one or more ports are configured to accommodate a temperature sensor.

Clause 44: The wet cell lid of clause 39, further comprising a window hole.

Clause 45: The wet cell lid of clause 44, wherein the window hole comprises glass.

Clause 46: The wet cell lid of clause 45, wherein the glass comprises a diameter of approximately 26 millimeters and a thickness of approximately 6 millimeters.

Clause 47: The wet cell lid of clause 45, wherein the one or more slots are configured to allow the air to escape a surface of the glass.

Clause 48: The wet cell lid of clause 39, further comprising one or more edges configured to aid in manipulating the wet cell lid.

Clause 49: The wet cell lid of clause 48, wherein the one or more edges comprise a proud edge.

Clause 50: An insert for holding an intraocular lens (IOL) in a wet cell, the insert comprising: a first side; a second side configured to removably engage with the first side; one or more standoffs configured to create a gap between the first and second sides when the first and second sides are engaged; one or more haptic pegs configured to constrain the IOL in a horizontal plane; and one or more lobes configured to reduce rotation of the insert when placed within the wet cell, wherein the insert is configured to hold the IOL between the first and second sides.

Clause 51: The insert of clause 50, where the first side of the insert comprises stainless steel.

Clause 52: The insert of clause 50, wherein the second side of the insert comprises polyetheretherketone (PEEK).

Clause 53: The insert of clause 50, wherein the insert further comprises one or more first chamfers configured to aid in directing water around the IOL.

Clause 54: The insert of clause 53, wherein the one or more first chamfers are further configured to allow air to escape from around the IOL.

Clause 55: The insert of clause 50, wherein the insert further comprises one or more second chamfers configured to aid in manipulating the insert.

Clause 56: The insert of clause 50, wherein the insert further comprises one or more posts configured to align the first and second sides when the first and second sides are engaged.

Clause 57: The insert of clause 50, wherein the gap prevents damage to the IOL.

Clause 58: The insert of clause 50, wherein the one or more lobes are configured to align with one or more recesses of a wet cell base of the wet cell to reduce the rotation of the insert.

Clause 59: The insert of clause 50, wherein first and second sides of the insert are configured to prevent floating of the insert inside the wet cell when the wet cell holds a liquid.

Clause 60: An assembly for transporting a wet cell comprising: an interface configured to removably engage with the wet cell and an insulating layer; and the insulating layer, wherein the assembly is configured to reduce the conduction of heat through the wet cell from an external material handling system.

Clause 61: The assembly of clause 60, wherein the interface comprises stainless steel.

Clause 62: The assembly of clause 60, wherein the interface comprises one or more chamfers configured to aid in engaging the interface with the wet cell.

Clause 63: The assembly of clause 60, wherein the insulating layer comprises one or more offset ribs configured to reduce the conduction of heat through the wet cell from the external material handling system.

Clause 64: The assembly of clause 60, wherein the insulating layer comprises polyetheretherketone (PEEK).

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. A system for use in automated intraocular lens metrology, the system comprising:
a wet cell comprising:
a wet cell base comprising one or more first recesses;
a wet cell lid; and
an insert configured for placement within the wet cell base and comprising:
a first side;
a second side configured to removably engage with the first side;
one or more first components configured to align with the one or more first recesses to inhibit rotation of the insert within the wet cell base; and
one or more second components configured to constrain an intraocular lens (IOL) in a horizontal plane,
wherein the insert is further configured to hold the IOL between the first and second sides; and
an assembly configured to removably engage with and reduce the conduction of heat through the wet cell.

2. The system of claim 1, wherein the first side of the insert comprises stainless steel.

3. The system of claim 1, wherein the second side of the insert comprises polyetheretherketone (PEEK).

4. The system of claim 1, wherein the insert comprises one or more standoffs configured to create a gap between the first and second sides when the first and second sides are engaged.

5. The system of claim 1, wherein the one or more second components comprise one or more haptic pegs.

6. The system of claim 1, wherein the insert comprises one or more posts configured to align the first and second sides when the first and second sides are engaged.

7. The system of claim 1, wherein the one or more first components comprise one or more lobes, and wherein the second side of the insert comprises the one or more lobes.

8. The system of claim 1, wherein the assembly is configured to reduce the conduction of heat through the wet cell from an external material handling system.

9. The system of claim 1, wherein:
the assembly is configured to removably engage with the wet cell base.

10. The system of claim 1, wherein the assembly comprises one or more offset ribs configured to reduce the conduction of heat through the wet cell.

11. A system for use in automated intraocular lens metrology, the system comprising:
a wet cell configured to hold a liquid and comprising:
a wet cell base comprising one or more first recesses;
a wet cell lid; and
an insert configured for placement within the wet cell base and comprising:
a first side;
a second side configured to removably engage with the first side;
one or more first components configured to align with the one or more first recesses to inhibit rotation of the insert within the wet cell base; and
one or more second components configured to constrain an intraocular lens (IOL) in a horizontal plane,
wherein the insert is configured to hold the IOL between the first and second sides when the first and second sides are engaged; and
wherein the weight of the first side is greater than the weight of the second side to prevent floating of the insert inside the wet cell when the wet cell holds the liquid.

12. The system of claim 11, wherein the first side of the insert comprises stainless steel.

13. The system of claim 12, wherein the second side of the insert comprises polyetheretherketone (PEEK).

14. The system of claim 11, wherein the insert comprises one or more standoffs configured to create a gap between the first and second sides when the first and second sides are engaged.

15. The system of claim 11, wherein the one or more second components comprise one or more haptic pegs.

16. The system of claim 11, wherein the insert comprises one or more posts configured to align the first and second sides when the first and second sides are engaged.

17. The system of claim 11, wherein the one or more first components comprise one or more lobes, and wherein the second side of the insert comprises the one or more lobes.

18. A method for conducting automated intraocular lens metrology, the method comprising:
engaging an intraocular lens (IOL) with a first side of an insert;
engaging a second side of the insert with the first side such that the IOL is held between the first and second sides, wherein the insert comprises one or more first components configured to constrain the IOL in a horizontal plane;
placing the insert into a wet cell base of a wet cell such that the first side of the insert faces a first direction within the wet cell base and the second side of the insert faces a second direction within the wet cell base, restricting the movement of the IOL within the wet cell by the configuration of the insert;
engaging a wet cell lid with the wet cell base; and
automatically measuring one or more features of the IOL.

19. The method of claim 18, wherein:
the first direction comprises an upward direction;
the second direction comprises a downward direction; and
the weight of the second side is greater than the weight of the first side to prevent floating of the insert inside the wet cell when the wet cell holds a liquid.

20. The method of claim 19, further comprising:
engaging the wet cell base with an assembly configured to reduce the conduction of heat through the wet cell from an external material handling system.
